(19) United States
(12) Patent Application Publication
(10) Pub. No.: US 2011/0077294 A1
(43) Pub. Date: Mar. 31, 2011

(54) R-(-) / S-(+)-7-[3-N SUBSTITUTED AMINO-2 HYDROXYPROPOXY] FLAVONES

(76) Inventors: Ram Pratap, Uttar Pradesh (IN); Himanshu Singh, Uttar Pradesh (IN); Alok Kumar Verma, Uttar Pradesh (IN); Amar Bahadur Singh, Uttar Pradesh (IN); Priti Priti Tiwari Tiwari, Uttar Pradesh (IN); Mukesh Srivastava, Uttar Pradesh (IN); Arvind Kumar Srivastava, Uttar Pradesh (IN); Anil Kumar Dwivedi, Uttar Pradesh (IN); Satyawan Singh, Uttar Pradesh (IN); Pratima Pnahma Svivastva Srivastava, Uttar Pradesh (IN); Shio Kumar Singh, Uttar Pradesh (IN); Chandishwar Nath, Uttar Pradesh (IN); Ram Raghubir, Uttar Pradesh (IN)

(21) Appl. No.: 12/376,909

(22) PCT Filed: Aug. 2, 2007
(86) PCT No.: PCT/IN07/00326

§ 371 Date: Apr. 19, 2010

(30) Foreign Application Priority Data
Aug. 9, 2006 (IN) 1803/DEL/2006

(51) Int. Cl. A61K 31/352; C07D 311/60; A61P 3/10; A61P 3/06

(52) U.S. Cl. 514/456; 549/403

(57) ABSTRACT

The present invention provides an optically active compound of general formula (I) and salts thereof: wherein R′ is selected from a group consisting of t-butyl amine, n-butylamine, iso-butylamine, iso-propyl amine, 4-phenyl-piperazine-1-ylamine, 4-(2-methoxyphenyl)-piperazin-1-ylamine, and 3,4-dimethoxy phenethyl amine; wherein R₁, R₂ and R₃ are selected from the group consisting of hydrogen, methyl or iso pentenyl; R₄, R₅ and R₆ are selected from the group consisting of Oil, O-alkyl, O-benzyl, O-substituted phenyl or combination thereof.

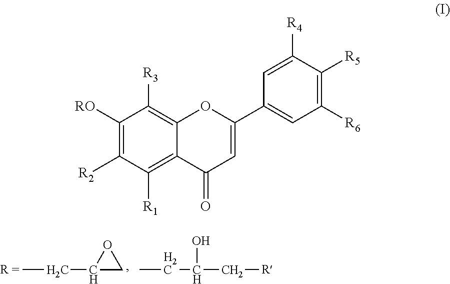

### FIELD OF INVENTION

The present invention relates to R (−)/S-(+)-7-[3-N-substituted amino-2-hydroxypropoxy] flavones and salts thereof. The present invention relates to synthesis of suitably substituted flavone derivatives exhibiting pronounced antidiabetic and antidyslipidemic activities. More particularly the invention relates to synthesis of R (−)/S-(+)-7-[3N-substitutedamino-2-hydroxypropoxy] flavones of formula I and pharmaceutical composition containing these derivatives, as described in the following description.

### BACKGROUND OF THE INVENTION AND PRIOR ART

Type II insulin resistant diabetes mellitus accounts for 90-95% of all diabetes. Changed sedentary life style has contributed towards affliction of the disease to adult population also the main force driving this increasing incidence is a staggering increase in obesity, the single most important contributor to the pathogenesis of diabetes mellitus. Prolonged disease condition leads to chronic macrovascular complications such as retinopathy and nephropathy. The disease is collectively referred, as metabolic syndrome encompasses type II diabetes and common constellation of closely linked clinical features. Characteristic factors include insulin resistance per se, obesity, hypertension and a common form of dyslipidemia and low high-density lipoprotein cholesterol. Metabolic syndrome is associated with marked increased incidence of coronary, cerebral and peripheral artery disease [Executive summary of the third report of the National Cholesterol Program Expert Panel on detection, evaluation and treatment of high blood cholesterol in adults (2001), J. Am. Med. Assoc. 285, 2486-2496].

The role of peripheral and hepatic insulin resistance in the pathogenesis of diabetes mellitus is undisputed. Insulin resistance can be due to multiple defects in signal transduction such as impaired activation of insulin receptor-tyrosine kinase and reduced activation of insulin-stimulated phosphatidyl inositol-3-hydroxy kinase. The resistance of insulin due to diet-induced obesity [Elchebly, M. et al. (1999), Science, 283, 1544.] has given the critical role of obesity in the development of insulin resistance and other features of the metabolic syndrome. Successful approaches attenuating appetite and/or enhancing energy expenditure will prove to be of great benefit in preventing and treating type II diabetes. Abnormalities of fatty acid metabolism are increasingly recognized as key components of the pathogenesis of the metabolic syndrome and type II diabetes. A critical player in potentiating the promoting effect of hyperinsulinaemia on hepatic lipid accumulation is the anabolic transcription factor SREBP-1, which upregulates genes such as that for fatty acid synthase [Shimomura, I. et al. (2000), Mol. Cell, 6, 77-86.]. These observations support a unified “lipotoxicity” hypothesis, which states that metabolic syndrome and type II diabetes can be caused by the accumulation of triglycerides and long chain fatty-acyl-CoA in liver and muscle. The third causal factor of metabolic syndrome is oxidative stress. Excess levels of oxygen in the living body can also pose a serious health threat; the so-called oxygen toxicity is brought about by oxygen species such as hydrogen peroxide and oxy radicals and damage living tissue. The active oxygen species are associated with diabetes mellitus and are destructive towards various tissues as occurring in diabetes mellitus. There have been many reports discussing relationships between peroxidation and diseases such as diabetes mellitus, atherosclerosis and myocardial ischemia in terms of radical oxidation. Glucose is oxidized under oxidative stress to highly reactive species, which ultimately reacts with proteins. Glucose, like other α-hydroxy aldehydes, can enolize and thereby reduce molecular oxygen under physiological conditions, catalyzed by transition metals, yielding α-keto aldehydes and oxidizing intermediates. These secondary compounds are more reactive than monosaccharides and can react with proteins to form cross-linked Maillard products (Simon P. Wolff et al. 1991; Free Radical Biology and Medicine, 10, 339-352.).

Oxidative stress also modifies lipids. Like glucose, LDL also undergoes oxidative modification to form modified LDL (oxidized LDL). The actual oxidation process is believed to begin with lipid peroxidation, followed by fragmentation to give short chain aldehydes. These aldehydes in turn react with the lysine residues of apo-B, creating a new epitope, which is recognized by the scavenger receptor of macrophages. During this same process, lecithin is converted to lysolecithin, which is a selective chemotactic agent for monocytes. The monocytes enter the subendothelium and undergo a phenotypic change to a macrophage, which avidly take up the oxidized LDL via the scavenger receptor. The uptake of oxidized LDL continues until the macrophage is so engorged with cholesteryl esters that it transforms into a foam cell. Groups of these foam cells constitute a fatty streak, the earliest hallmark of atherosclerosis. By inhibiting the oxidation of LDL, it is hoped that the modification of apo-B and the production of chemotactic lysolecithin can be prevented and in-turn the atherosclerosis.

At present, therapy for type II diabetes relies mainly on several approaches intended to reduce the hyperglycemia itself: sulphonylureas which increase insulin secretion from pancreatic beta cells; Metformin which acts to reduce hepatic glucose production, peroxisome proliferator activated receptor-γ agonists which enhance insulin action and α-glucosidase inhibitors interfere with gut glucose absorption. These therapies have limited efficacy, limited tolerability and mechanism-based toxicity. Of particular concern is the tendency for most treatments to enhance weight gain. A problem particular to the sulphonylureas is that many patients who respond initially become refractory to treatment overtime.

The increasing prevalence of obesity and its associated co-morbidities including type II diabetes and related cardiovascular disorders has stimulated efforts to develop effective new approaches in the treatment of this condition. While most therapeutic approaches involve altering the balance of metabolic energy by reducing energy intake, an alternative approach for the management of obesity is to affect an increase in the rate of energy expenditure. In 1984, compounds of the phenethanolamine class as shown below having thermogenic properties in rodents were first disclosed. Despite their structural similarity to known β₁ and β₂ adrenoceptor ligands, pharmacological studies indicated that these compounds stimulated a third or ‘atypical’ β-adrenergic receptor (β-AR) that is now described as β₃-AR. β₃ agonist also increased insulin sensitivity and glucose utilization. Later studies suggested that Tyr 64 Arg β₃-AR mutation in the human population plays a role in the development of diabetes mellitus and/or obesity in some individuals possessing this genetic variant [Turner, N. C.; (1996), DDT, 1, 109-116]. Among phenethanolamine compounds as thermogenic agent acting on β₃-adrenergic receptor, CL-316,243 is exhibiting promising activity in humans.

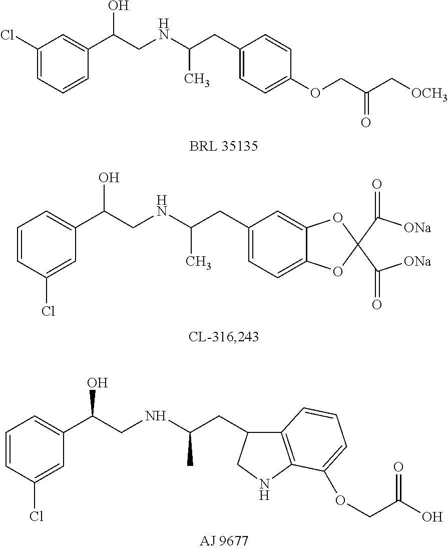

The paradigm of current drug design has shifted from single target to multi target drug called network models which suggest that partial inhibition of a surprisingly small number of targets can be more efficient than the complete inhibition of a single target. Troglitazone is such a drug which had pharmacophores to focus two targets however it failed because of its toxic metabolites. In order to design drug for metabolic syndrome X we targeted oxidative stress and stimulation of thermogenesis. We tried to amalgamate the pharmacophore for above targets in a single molecule. Flavonoids were selected as basic skeleton to attach phamacophore for thermogenesis. Flavonoids are among the most ubiquitous groups of polyphenolic compounds in foods of plant origin. As integral constituents of the diet, they may exert a wide range of beneficial effects on human health. Flavonoids produce such biological effects through their free radical scavenging antioxidant activities and metal ion chelating abilities. (Cotelle, N. et al, Free Rad. Biol. Med. 1992, 13, 211). These properties led us to utilize flavones for the synthesis of hybrid molecules as antidiabetic and antidyslipidemic agents by substitution with thermogenic as well as insulin sensitizing pharmacophores. In our earlier application we have made claim for 7-N substituted 2-hydoxy propoxy flavone as potent antilipidemic and antidyslipidemic agent. Here we claim antidiabetic and antidyslipidemic activity of their optical enantiomers.

### OBJECTS OF THE PRESENT INVENTION

The main objective of the present invention is to provide R (−)/S-(+)-7-[3-N-substituted amino-2-hydroxypropoxy]flavones and salts thereof.

Another object of the present invention is to provide process for preparation of compound of formula I.

Yet another object of the present invention is to provide a pharmaceutical composition containing these flavone derivatives and a pharmaceutically acceptable carrier or diluent thereof.

Yet another object of the present invention is to provide a method for treating type II diabetes and associated hyperlipidemic conditions in a mammal by administering a pharmaceutically acceptable amount of compound of formula I optionally along with pharmaceutically acceptable excipents.

### SUMMARY OF THE INVENTION

Accordingly, the present invention provides novel flavone derivatives of formula I which exhibit antihyperglycemic and antidyslipidemic activity. The invention also provides a method for controlling ‘type II’ diabetes and associated hyperlipidemic conditions in a mammal by administering these compounds and compositions containing these derivatives.

### DETAILED DESCRIPTION OF THE INVENTION

Accordingly, the present invention provides an optically active compound of general formula I and salts thereof:

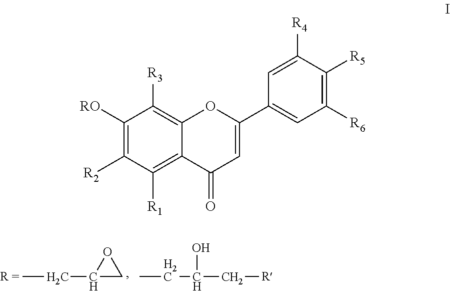

wherein R′ is selected from a group consisting of t-butyl amine, n-butylamine, iso-butylamine, iso-propyl amine, 4-phenyl-piperazine-1-ylamine, 4-(2-methoxyphenyl)-piperazin-1-ylamine, and 3,4-dimethoxy phenethyl amine;
wherein R₁, R₂ and R₃ are selected from the group consisting of hydrogen, methyl or iso pentenyl; R₄, R₅ and R₆ are selected from the group consisting of OH, O-alkyl, O-benzyl, O-substituted phenyl or combination thereof.

Another embodiment of the present invention provides a pharmaceutical composition comprising the compound of formula I and pharmaceutically acceptable quantities of a conventional pharmaceutically acceptable carrier or diluent thereof.

In yet another embodiment of the present invention, the compound of formula I is used along with pharmaceutically acceptable quantities of conventional lipid lowering agents and/or conventional sugar lowering agents.

Yet another embodiment of the present invention provides a method for treating type II diabetes and associated hyperlipidemic conditions in mammals by administering a pharmaceutically effective amount of compound of formula I, optionally with other conventional diabetic and lipid lowering agents.

Yet another embodiment of the present invention provides a method of treating macrovascular conditions such as retinopathy and nephropathy in mammals by administering a pharmaceutically effective amount of the compound of formula I, optionally with other conventional diabetic and lipid lowering agents.

In yet another embodiment of the present invention, the range of pharmaceutically effective dose is 10-250 mg/Kg body weight of the compound.

Still another embodiment of the present invention provides a compound of formula (4) of configuration R.

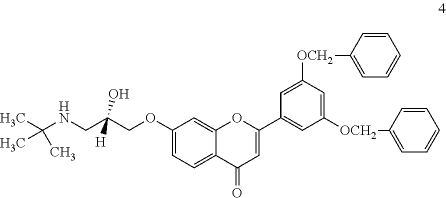

Still another embodiment of the present invention provides a compound of formula (7) of configuration S.

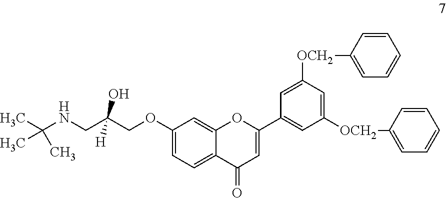

Yet another embodiment of the present invention provides a compound of formula (9) of configuration R.

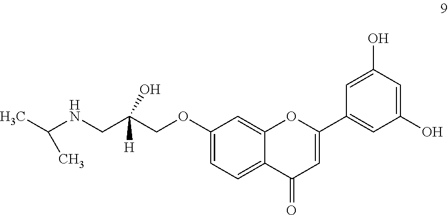

Yet another embodiment of the present invention provides a compound of formula (II) of configuration S.

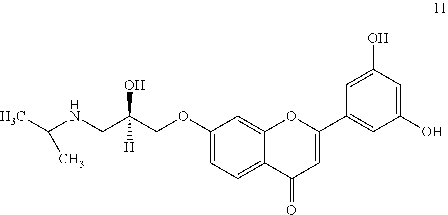

Still another embodiment of the present invention provides a compound having formula I or a pharmaceutically acceptable salt thereof, wherein the representative compounds are:
a) R-(−)-7-(3-tert-Butylamino-2-hydroxy-propoxy)-3′,5′-dibenzyloxy-flavone (4);
b) S-(+)-7-(3-tert-Butylamino-2-hydroxy-propoxy)-3′,5′-dibenzyloxy-flavone (7)
c) R-(−)-7-(3-iso-propylamino-2-hydroxy-propoxy)-3′,5′-dibenzyloxy-flavone (8);
d) R-(−)-7-(3-iso-propylamino-2-hydroxy-propoxy)-3′,5′-dihydroxy-flavone (9);
e) S-(+)-7-(3-iso-Propylamino-2-hydroxy-propoxy)-3′,5′-dibenzyloxy-flavone (10);
f) S-(+)-7-(3-iso-Propylamino-2-hydroxy-propoxy)-3′,5′-dihydroxy-flavone (11).

Accordingly the present invention provides a process for preparation of compound of general formula I, comprising the steps of:
(i) reacting substituted hydroxyflavone of general formula A wherein R₁, R₂ and R₃ are selected from the group consisting of hydrogen, methyl or iso pentenyl; wherein R₄, R₅ and R₆ are selected from the group consisting of O-alkyl, O-benzyl, O-substituted hydroxy, phenyl or combination thereof, with optically active R or S epi-chlorohydrin followed by reaction with a base to obtain the inverted 2,3-epoxy-propoxy-flavones of compound of formula B,

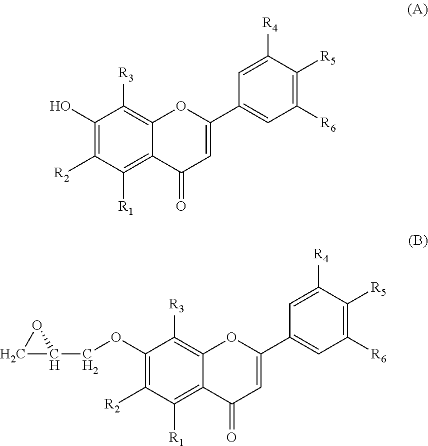

(ii) heating the 2,3-epoxy-propoxy-flavones of the formula B as obtained in step (i) under reflux, with an amine in an alcohol to yield propanolamines of formula C wherein R′ is selected from a group consisting of t-butyl amine, n-butylamine, iso-butylamine, iso-propyl amine, 4-phenyl-piperazine-1-ylamine, 4-(2-methoxyphenyl)-piperazin-1-ylamine, and 3,4-dimethoxy phenethyl amine,

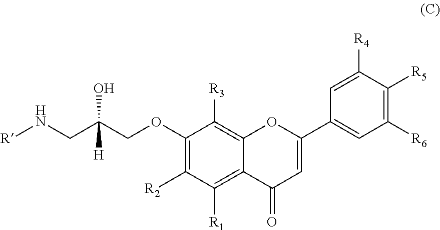

(iii) subjecting the propanolamine obtained in step (ii) for debenzylation using catalytic hydrogenation process to yield corresponding hydroxy flavone derived propanolamines of general formula C wherein R₄, R₅, and R₆ are selected from the group of H, OH and O-methoxy.

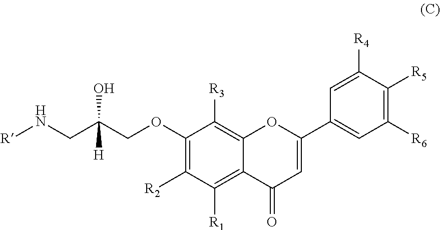

(iv) converting the compound of formula C into salts by known method.

In an embodiment of the invention, the base is selected from the group consisting of sodium hydroxide, potassium hydroxide and benzyltriethylammonium hydroxide.

In another embodiment of the invention, the amine used is selected from the group consisting of alkylamines from a group of cyclic and acyclic alkyl part.

In another embodiment of the invention, the alcohol used is selected from the group consisting of ethanol and propanol.

In another embodiment of the invention, the debenzylation is carried out using Pd—C as catalyst under hydrogen pressure ranging between 40-100 psi.

In another embodiment of the invention, the salt of general formula is selected from the group consisting of hydrochloride, fumarate and tartrates

Accordingly the present invention provides a method of treating type II diabetes and dislipidemia in mammals, said method comprising the step of administering pharmaceutically effective amount of compound of formula I, and salts thereof, optionally with the pharmaceutically acceptable exciepients.

In an embodiment of the invention, the pharmaceutically effective amount of compound of formula I is in the range of 10-100 mg./kg-body weight.

In another embodiment of the invention, the subject may be selected from animals including human.

In another embodiment of the invention, the compound of formula I showed maximum fall in blood glucose was recorded to be around, 61.1% at a dose around 25 mg/kg body weight.

In a further embodiment of the invention, the compound (S)-(+)-7-(3-tert-butylamino-2-hydroxypropoxy)-3′,5′-dibenzyloxyflavone (7) showed an elevation of 33.2% was measured in HDL-cholesterol level than the control ones at dose around 25 mg/kg body weight.

In yet another embodiment of the invention, ED₅₀ of compound of general formula I is in the range of 17.84 to 32.93 mg/kg.

In another embodiment of the invention, the compound (S)-(+)-7-(3-tert-butylamino-2-hydroxypropoxy)-3′,5′-dibenzyloxyflavone (7) enhances HDL,C more than two folds as compared to the racemic compound at a dose of 25 mg/kg body weight.

### Chemistry:

Aryloxypropanolamines are well established pharmacophore for β-adrenergic receptors and therefore various enantioselective syntheses for example, using chiral glycerol-1,2-acetonide [Danilewicz J. C., Kemp .J. E. G., J. Med. Chem., 16168 (1973)], glycidylarenesulfonate [Klunder J. M., Onami T. Sharpless K. B., J. Org. Chem 54, 1295 (1989)] or epi-chlorohydrin [Baldwin J. J., Raab A. W., Mensler K., Arison B. H., McClure D. E., J. Org. Chem. 43, 4876 (1978)] and [Seki T., Kanada A., Nakao T., Shiraiwa M., Asano H., Miyazawa K., Ishimori T., Minami. N., Shibata K., Yasuda K., Chem Pharma Bull 46(1), 84 (1998)] are known in literature. A phenoxide anion reacts with glycidyl derivative either at C-1 or C-3 depending on the reactivity substituent at allylic position of oxyrane. The attack at C-1 causes direct displacement of X (path a) in case of arenesulfonate whereas that at C-3 in case of epi-chlorohydrin causes epoxide ring opening, which following internal displacement of the leaving group X (path b) as shown in Fig I results the product of opposite configuration to path a. We synthesized the optical isomers of our product following path b.

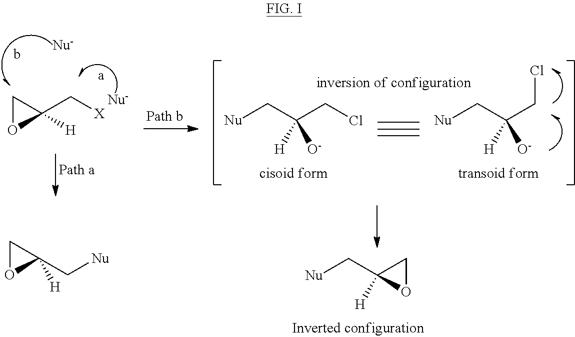

Therefore when the reaction of flavone was carried out with (R)-epi-chlorohydrin at 120° C. without the solvent, the intermediate chlorohydrin was obtained in an excellent yield. The chlorohydrin without purification taken in toluene and treated with aqueous NaOH in presence of catalytic amount of phase transfer catalyst triethylbenzylanimonium chloride (TEBA) to give S-isomer in 79% yield with 95% ee. Thus the nucleophilic substitution reaction at 120° C. proceeded at the terminal C atom of the epoxide ring with high regioselectivity, probably via activating complexation (Fig II) as presumed by McClure D. E., Arison B. H., Baldwin J. J., J. Am. Chem. Soc., 101, 3666(1979).

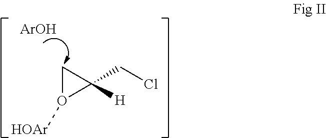

The epoxide thus obtained was treated with appropriate amine in methanol to yield desired amino propanol derivative. The purity of these compounds was ascertained by HPLC on comparison of the racemic mixture graph with that of the enantiomers graph. The HPLC study was performed on Chira Sphere NT (250 mm×0.4 mm, 5 μm, Merck) column using a mixture of solvent hexane: iso-propanol: methanol: ammonia (in a ratio of 60:40:5:0.5 ml) at the flow rate of 2 ml/min and peak detection at 240 mm under UV. The graph of one of the mixtures and its enantiomers are given in figure III.

### EXAMPLES

(R)-(−)-7-(3-tent-Butylamino-2-hydroxypropoxy)-3′,5′-dibenzyloxyflavone (4, S002-853 R, Fig IV):

A mixture of 3′,5′-dibenzyloxy-7-hydroxy flavone (1, 1.0 gm, 2.2 mmol) and (S)-epi-chlorohydrin (1.0 ml, 12.0 mmol) was stirred for about 2 hrs at 120° C. The cooled reaction mixture was taken in toluene and to this was added dropwise a solution of NaOH (10 mg, 0.25 mmol) and benzyltriethyl ammonium chloride (10 mg, 0.04 mmol) in water with stirring at room temperature for 2 hrs, and the organic layer was separated, dried over sodium sulphate and solvent evaporated under reduced pressure to give crude 3′,5′-dibenzyloxy-7-(2,3-epoxy-propoxy) flavone (3) of R-configuration. Yield (900 mg, 80%)

The above (R)-3′,5′-dibenzyloxy -7-(2, 3-epoxy-propoxy) flavone (600 mg, 4.9 mmol) and tert-butyl amine (0.26 ml, 9.8 mmol) in dry methanol (50 ml) was stirred at reflux for about 6 hrs. The solvent was evaporated at reduced pressure and the crude product was purified by column chomatography to afford (R)+) 7-(3-tent-butylamino-2-hydroxypropoxy)-3′,5′-dibenzyloxyflavone (4) Yield (650 mg, 93%); m.p 158° C.; [α]_{D}=−8.6 (c=1.5%, CHCl₃).

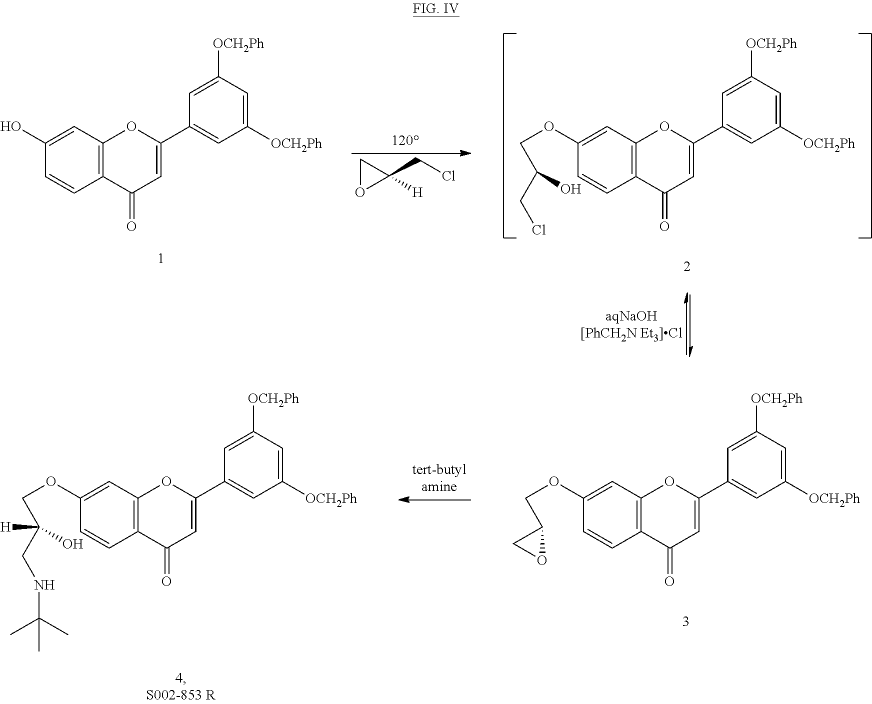

(S)-(+)-7-(3-tert-Butylamino-2-hydroxypropoxy)-3′,5′-dibenzyloxyflavone (7, S002-853 S, Fig V):

A mixture of 3′,5′-dibenzyloxy-7-hydroxy flavone (1) (1.2 g, 2.6 mmol) and (R)-epi-chlorohydrin (1.12 ml, 15.0 mmol) was stirred for about 2 hrs at 120° C. The cooled reaction mixture was taken in toluene and to this was added a solution of NaOH (15 mg, 0.37 mmol) and benzyltriethylammoniumchloride (15 mg, 0.06 mmol) in water, stirred at room temperature for 2 hrs. The organic layer was separated, dried over sodium sulphate and solvent removed under reduced pressure to give crude (S)-(+)-7-(2,3-epoxy-propoxy)-3′,5′-dibenzyloxy flavone (6). Yield (1.12 g, 83%)

The solution of above (S)-3′,5′-dibenzyloxy -7-(2,3-epoxy-propoxy)-flavone (500 mg, 4.9 mmol) in dry methanol (50 ml) and tent-butyl amine (0.2 ml, 9.8 mmol) was stirred at reflux for about 6 hrs. The solvent removed at reduced pressure and crude product was purified by column chromatography to afford (S)-(+)-7-(3-tert-butylamino-2-hydroxy-propoxy)-3′,5′-dibenzyloxy-flavone (7), Yield (520 mg, 93%); m.p165°; [α]_{D}=+9.0 (c=20 mg/7.5 ml of CHCl₃).

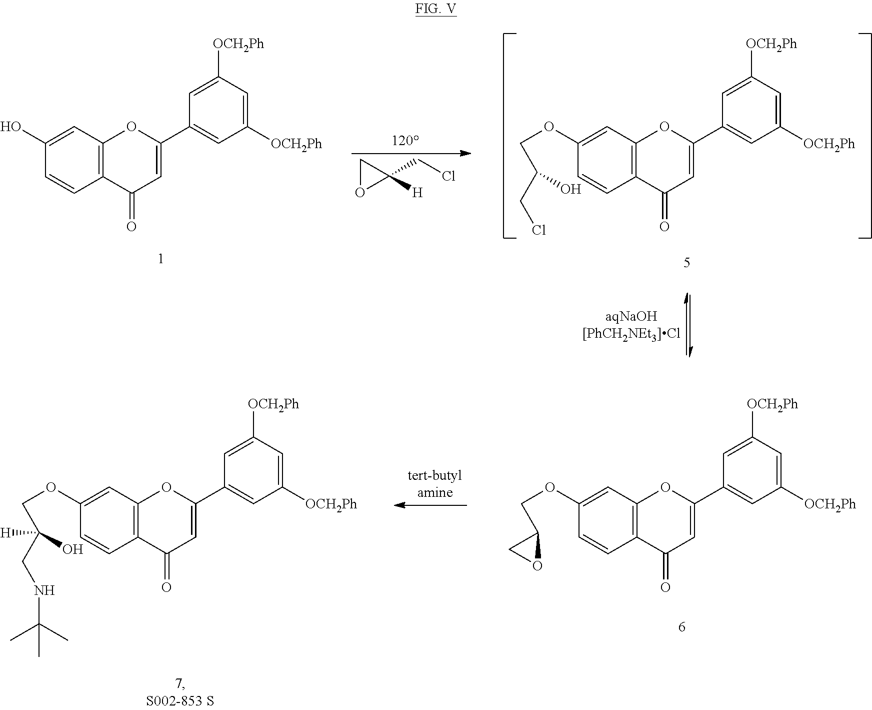

(R)-(−)-7-(2-Hydroxy-3-isopropyl aminopropoxy)-3′,5′-dihydroxy flavone (9, S002-857 R, Fig VI):

A mixture of 3′,5′-dibenzyloxy-7-hydroxy-flavone (1) (1 g, 2.2 mmol) and S-epi-chlorohydrin (1.0 ml, 12.0 mmol) was stirred for about 2 hrs at 120 ° C. The cooled mixture was taken in toluene and to this was added a solution of NaOH (10 mg, 0.25 mmol) and benzyltriethyl ammonium chloride (10 mg, 0.04 mmol) in water and stirred at room temperature for 2 hrs. The organic layer was separated, dried over sodium sulphate and evaporated under reduced pressure to give crude R-3′,5′-dibenzyloxy-7-(2,3-epoxy-propoxy)-flavone (3). Yield (900 mg, 80%)

The solution of above (R)-3′,5′-dibenzyloxy-7-(2,3-epoxy-propoxy)-flavone (600 mg,4.9 mmol) and iso-propylamine (0.5 ml, 14.7 mmol) in dry methanol (50 ml) was stirred at reflux for about 6 hrs. The solvent was removed at reduced pressure and the crude product so obtained was purified by column chomatography to give (R) 7-(2-hydroxy-3-isopropyl amino-propoxy)-3′,5′-dibenzyloxy flavone (8) Yield (550 mg, 79%)

(R)-3′,5′-Dibenzyloxy-7-(2-hydroxy-3-isopropyl amino-propoxy) flavone (300 mg, 2.12 mmol) was shaken in dry methanol (40 ml) with 10% Pd/C (quantity sufficient) under hydrogen atmosphere. The catalyst was filtered and the solvent removed under reduced pressure to give the product (R)-(+)-7-(2-hydroxy-3-isopropylamino-propoxy)-3′,5′-dihydroxy flavone (9), Yield (200 mg, 98%) m.p; 124° C.; [α]_{D}=−9 (c=20 mg/7.5 ml of 2% CHCl₃/MeOH).

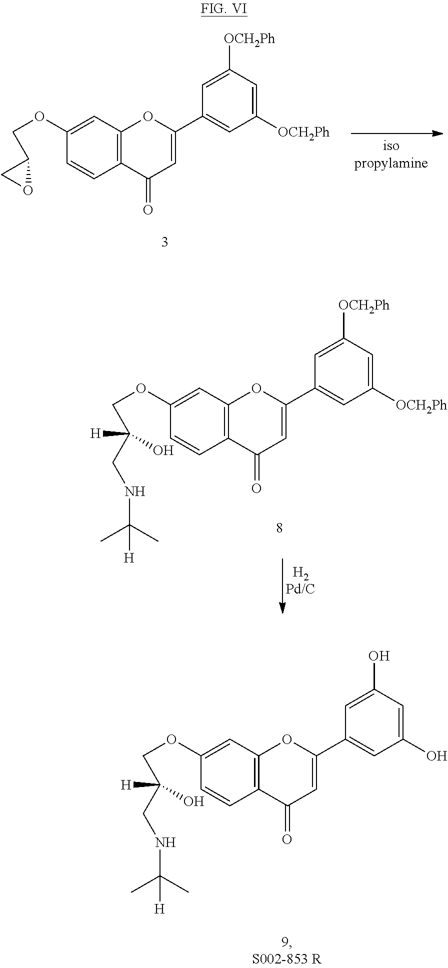

(S)-(+)-7-(2-Hydroxy-3-isopropyl aminopropoxy)-3′,5′-dihydroxyflavone (11, S002-857 S, Fig VII):

A mixture of 3′,5′-dibenzyloxy-7-hydroxyflavone (1.2 g, 2.6 mmol) and R-epi-chlorohydrin (1.2 ml, 15.0 mmol) was stirred for about 2 hrs at 120°. The cooled reaction mixture was taken in toluene and to this was added a solution of NaOH (15 mg, 0.37 mol) and benzyltriethyl ammonium chloride (15 mg, 0.06 mmol) in water and stirred at room temperature for 2 hrs. The organic layer was separated, dried over sodium sulphate and solvent removed under reduced pressure to give crude S-3′,5′-dibenzyloxy-7-(2,3-epoxy-propoxy) flavone (6) Yield (1.12 g, 83%).

The solution of above (S) -7-(2,3-epoxy-propoxy)-3′,5′-dibenzyloxy-flavone (500 mg, 4.9 mmol) and iso-propylamine(0.3 ml, 14.7 mmol) in dry methanol (50 ml) was stirred at reflux for about 6 hrs. The solvent removed at reduced at reduced pressure the crude product so obtained was purified by column chomatography to afford (S) 7-(2-hydroxy-3-isopropyl amino-propoxy)-3′,5′-dibenzyloxy-flavone (10) Yield (440 mg, 79%).

(S)-7-(2-Hydroxy-3-isopropylamino-propoxy)-3′,5′-dibenzyloxy-flavone (250 mg, 2.12 mmol) in dry methanol (40 ml) and 10% Pd/C (sufficient amount) was shaken in hydrogen atmosphere at about 40 psi. The catalyst was filtered and solvent removed to give the product (S)-(+) -7-(2-hydroxy-3-iso-propylamino-propoxy)-3′,5′-dihydroxy-flavone (11) Yield (160 mg, 98%); m.p.130° C.; [α]_{D}=+15.4 (c=20 mg/7.5 ml of 2% CHCl₃/MeOH).

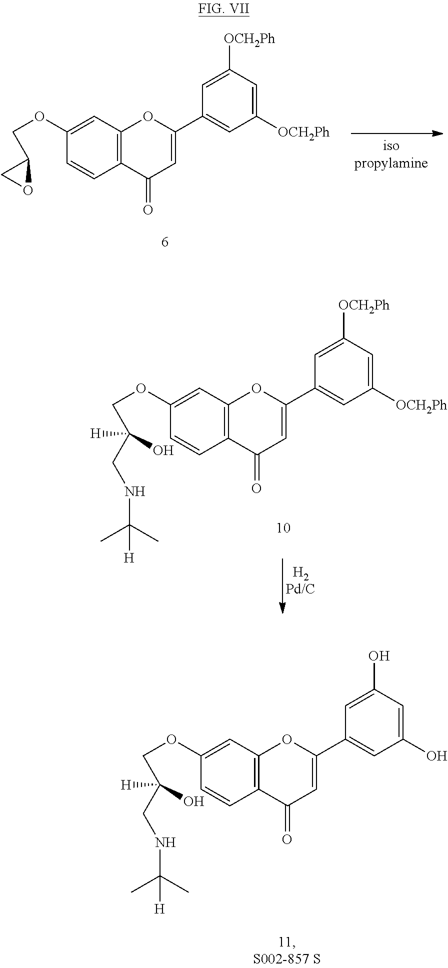

### Biological Evaluation:

### Experimental Protocol:

C57BL/KsBom-db/db mice (12-18 weeks old), weighing around 35+g bred in the animal house of CDRI, Lucknow, were used in the present experiments. The mice were housed in groups of 3 to 5 (same sex) in a room controlled for temperature (23±2.0 ° C.) and 12/12 hours light/dark cycle (lights on at 6.00 am). Body weight of each animal was measured daily from day 1 to day 10 of the experiment. All animals had free access to fresh water and normal pellet diet except on the day of the postprandial protocol (day 6) and during the overnight fast before the OGTT on day 10. The animals always had access to water during experimental periods. Blood glucose was checked every morning up till day 10. On day 10 an oral glucose tolerance test (OGTT) was performed after an overnight fasting. Blood glucose was measured at −30.0 min and test compounds were administered. The blood glucose was again measured at 0 min just post treatment and at this juncture glucose solution was given at a dose of 3 gm/kg to all the groups including the vehicle treated group. The blood glucose levels were checked at 30 min, 60 min, 90 min and 120 min post glucose administration. The data was analyzed for its significance on Prism software.

### Antihyperglycemic Activity Profile:

(S)-(+)-7-(3-tert-Butylamino-2-hydroxypropoxy)-3′,5′-dibenzyloxyflavone (7, S-002-853 S):

It is evident from the data that compound (7, S-002-853 S) exhibit significant antihyperglycemic and antidyslipidemic activity in db/db mice at 25 mg/kg dose. At this dose it showed maximum fall in blood glucose profile at day 6 of treatment as shown in the table 1. The maximum percentage fall in blood glucose was recorded to be around 61.1%. Fig VIII shows the graphical presentation of blood glucose profile versus days of treatment at 25 mg/kg dose level. Fig IX and table 2 presents effect of the 10 days treatment of the compound on glucose tolerance of the db/db mice. This effect was calculated to be around 45.3%.

Table 3 and Fig X presents the antidyslipidemic activity profile of (S)-(+)-7-(3-tert-butylamino-2-hydroxy propoxy)-3′,5′-dibenzyloxyflavone (7, S-002-853 S) in db/db mice at 25 mg/kg dose level. A lowering in the case of serum cholesterol and triglycerides by the treatment of (S)-(+)-7-(3-tert-butylamino-2-hydroxylpropoxy)-3′,5′-dibenzyloxyflavone (7, S-002-853 S) was recorded which was around 21.8% and 14.5%, respectively, at 25 mg/kg dose. An elevation of 33.2% was measured in HDL-cholesterol level than the control ones while a decrease of around 29.4% was measured in LDL-cholesterol level in (S)-(+)-7-(3-tert-butylamino-2-hydroxypropoxy)-3′,5′-dibenzyloxyflavone (7, S-002-853 S) treated db/db mice at 25 mg/kg dose.

TABLE 1
Blood glucose profile of sham treated and (S)-(+)-7-(3-tert-butylamino-2-
hydroxypropoxy)-3′,5′-dibenzyloxyflavone (7, S-002-853 S) treated db/db mice.

| | Blood glucose (mM) days post treatment | | | | | | | | |
|---|---|---|---|---|---|---|---|---|---|
| | 1 | 2 | 3 | 4 | 5 | 6 | 7 | 8 | 9 |
| Sham treated | 22.4 ± 1.69 | 25.0 ± 3.24 | 21.4 ± 5.07 | 23.6 ± 3.81 | 19.8 ± 4.63 | 19.4 ± 5.12 | 22.2 ± 5.91 | 20.8 ± 4.80 | 19.1 ± 4.10 |
| S-002-853 (S) | 22.4 ± 1.24 | 16.6 ± 3.85 | 12.9 ± 3.00 | 10.6 ± 2.89 | 8.80 ± 3.84 | 7.60 ± 3.46 | 10.6 ± 2.20 | 8.30 ± 1.13 | 7.90 ± 1.30 |
| treated | | | | | | | | | |
| (25 mg/kg) | | | | | | | | | |
| % change | +0.20 | −33.6 | −39.5 | −54.9 | −55.6 | −61.1 | −52.2 | −60.0 | −58.5 |
| compared to | | | | | | | | | |
| control | | | | | | | | | |
| Statistical | ns | ns | ns | p < 0.05 | p < 0.05 | p < 0.05 | p < 0.05 | p < 0.05 | p < 0.05 |
| significance | | | | | | | | | |

TABLE 2
Blood glucose profile of sham treated and (S)-(+)-7-(3-tert-butylamino-2-
hydroxypropoxy)-3′,5′-dibenzyloxyflavone (7, S-002-853 S) treated db/db mice.

| | Blood glucose profile (mM) min post glucose load | | | | | |
|---|---|---|---|---|---|---|
| | −30 | 0 | 30 | 60 | 90 | 120 |
| Sham treated | 8.09 ± 0.63 | 8.28 ± 0.63 | 29.0 ± 2.06 | 31.5 ± 0.91 | 23.8 ± 2.73 | 21.1 ± 3.36 |
| S-002-853 S treated | 4.57 ± 0.30 | 4.81 ± 0.35 | 17.8 ± 1.24 | 16.3 ± 1.82 | 13.0 ± 1.66 | 8.83 ± 0.72 |
| (25 mg/kg) | | | | | | |
| % change | −43.5 | −41.8 | −38.6 | −48.3 | −45.5 | −58.2 |
| compared to control | | | | | | |

TABLE 3
Serum lipid profile in sham treated and (S)-(+)-7-
(3-tert-butylamino-2-hydroxypropoxy)-3′,5′-
dibenzyloxyflavone (7, S-002-853 S) treated db/db mice.

| | Serum lipid profile (mg/dl) | | |
|---|---|---|---|
| | Triglycerides | Cholesterol | HDL-Chol |
| Sham treated | 135 ± 3.32 | 238 ± 24.6 | 37.2 ± 1.92 |
| S-002-8S3 S | 115 ± 2.82 | 185 ± 51.4 | 49.5 ± 8.36 |
| (25 mg/kg) | | | |
| % change | −14.5 | −21.8 | +33.2 |
| compared to control | | | |

− denotes decrease
+ denotes increase
Values are Mean ± SE of 6 animals per group

Tables 4 and 6 presents antihyperglycemic and antidyslipidemic activity profile of the db/db mice treated with (S)-(+)-7-(3-tert-butylamino-2-hydroxypropoxy)-3′,5′-dibenzyloxyflavone (7, S-002-853 S) at 10 mg/kg dose. Though, at this dose level it showed fall in blood glucose profile from day 3 of treatment and remained till the end of study. However, it was not statistically significant on any day post treatment. Fig XI shows the graphical presentation of blood glucose profile versus days of treatment at 10 mg/kg dose level. Table 5 and Fig XII presents effect of the 10 days treatment of the compound on glucose tolerance of the db/db mice. This effect was calculated to be around 27.6% but was not statistically significant. Table 6 and fig XIII presents the antidyslipidemic activity profile of(S)-(+)-7-(3-teat-butylamino-2-hydroxypropoxy)-3′,5′-dibenzyloxyflavone (7, S-002-853 S) in db/db mice at 10 mg/kg dose level. A lowering in the case of serum cholesterol and triglycerides by the treatment of (S)-(+)-7-(3-tert-butylamino-2-hydroxypropoxy)-3′,5′-dibenzyloxyflavone (7, S-002-853 S) was recorded which was around 16.3% and 17.9%, respectively, at 10 mg/kg dose. An elevation of +17.4% was measured in HDL-cholesterol level than the control ones. These difference, however, were statistically insignificant.

TABLE 4
Blood glucose profile of sham treated and (S)-(+)-7-(3-tert-butylamino-2-
hydroxypropoxy)-3′,5′-dibenzyloxyflavone (7, S-002-853 S) treated db/db mice.

| | Blood glucose (mM) days post treatment | | | | | | | | |
|---|---|---|---|---|---|---|---|---|---|
| | 1 | 2 | 3 | 4 | 5 | 6 | 7 | 8 | 9 |
| Sham treated | 19.7 ± 2.11 | 20.4 ± 2.84 | 21.9 ± 2.66 | 21.3 ± 2.21 | 22.1 ± 1.36 | 20.4 ± 3.55 | 20.8 ± 3.82 | 23.5 ± 3.88 | 12.4 ± 2.04 |
| S-002-853 (S) | 19.8 ± 2.47 | 18.5 ± 2.34 | 17.9 ± 2.14 | 15.3 ± 2.96 | 13.8 ± 2.98 | 13.1 ± 3.41 | 11.8 ± 3.55 | 12.1 ± 3.39 | 14.2 ± 2.98 |
| treated | | | | | | | | | |
| (10 mg/kg) | | | | | | | | | |
| % change | −0.9 | −9.60 | −18.6 | −26.8 | −35.0 | −40.7 | −42.0 | −42.2 | −39.4 |
| compared to | | | | | | | | | |
| control | | | | | | | | | |
| Statistical | ns | ns | ns | ns | ns | ns | ns | ns | ns |
| significance | | | | | | | | | |

TABLE 5
Blood glucose profile of sham and (S)-(+)-7-(3-tert-butylamino-2-hydroxypropoxy)-
3′,5′-dibenzyloxyflavone (7, S-002-853 S) treated db/db mice post oral glucose load.

| | Blood glucose profile (mM) min post glucose load | | | | | |
|---|---|---|---|---|---|---|
| | −30 | 0 | 30 | 60 | 90 | 120 |
| Sham treated | 11.0 ± 2.02 | 12.4 ± 2.04 | 28.7 ± 2.67 | 25.3 ± 3.89 | 16.6 ± 2.45 | 10.4 ± 0.43 |
| S-002-853 (S) treated | 8.37 ± 2.98 | 9.23 ± 3.10 | 19.3 ± 5.52 | 18.4 ± 5.39 | 12.1 ± 3.81 | 9.23 ± 3.10 |
| (10 mg/kg) | | | | | | |
| % change | −23.9 | −25.7 | −32.8 | −27.2 | −26.9 | −11.7 |
| compared to control | | | | | | |

TABLE 6
Serum lipid profile in sham treated and (S)-(+)-7-
(3-tert-butylamino-2-hydroxypropoxy)-3′,5′-
dibenzyloxyflavone (7, S-002-853 S) treated db/db mice.

| | Serum lipid profile (mg/dl) | | |
|---|---|---|---|
| | Triglycerides | Cholesterol | HDL-Chol |
| Sham treated | 134 ± 12.7 | 104 ± 8.27 | 56.0 ± 2.45 |
| S-002-853 (S) | 110 ± 4.08 | 87.5 ± 3.00 | 66.4 ± 2.87 |
| (10 mg/kg) | | | |
| % change | −17.9 | −16.3 | +17.4 |
| compared to control | | | |

− denotes decrease
+ denotes increase.
Values are Mean ± SE of 4 animals per group

(R)-(+7-(3-tert-Butylamino-2-hydroxypropoxy)-3′,5′-dibenzyloxyflavone (4, S-002-853 R):

Tables 7 and 9 present antihyperglycemic and antidyslipidemic activity profile of the db/db mice treated with (4, S-002-853 R) at 25 mg/kg dose. At this dose level, though the blood glucose profile of S-002-853 (R) treated mice was lowered from day 3 and it remained the same through the period of study. However, there was found no significant difference in the blood glucose profile between the two groups at any day. Fig XIV shows the graphical presentation of blood glucose profile versus days of treatment at 25 mg/kg dose level. There was also found no difference in oral glucose tolerance curve between the sham treated and S-002-853 (R) treated db/db mice. (Table 8; Figure XV). Table 9 presents the lipid profile of S-002-853 R and sham treated groups. There was found no significant lowering in either the case of serum cholesterol or triglycerides by the treatment of (R)-(−)-7-(3-tert-butylamino-2-hydroxypropoxy)-3′,5′-dibenzyloxyflavone (4, S-002-853, R). There was also found no significant difference in the HDL-cholesterol levels between the sham and S-002-853 (R) treated groups (Table 9, fig XVI).

TABLE 7
Blood glucose profile of sham treated and (R)-(−)-7-(3-tert-butylamino-2-
hydroxypropoxy)-3′,5′-dibenzyloxyflavone (4, S-002-853 R) treated db/db mice.

| | Blood glucose (mM) days post treatment | | | | | | | | |
|---|---|---|---|---|---|---|---|---|---|
| | 1 | 2 | 3 | 4 | 5 | 6 | 7 | 8 | 9 |
| Sham treated | 11.8 ± 3.31 | 12.2 ± 3.56 | 15.1 ± 5.26 | 17.5 ± 2.08 | 15.0 ± 0.45 | 15.9 ± 0.46 | 18.1 ± 1.62 | 19.7 ± 0.75 | 20.8 ± 2.06 |
| S-002-853 (R) | 12.0 ± 2.52 | 12.4 ± 2.65 | 10.3 ± 1.33 | 13.3 ± 4.61 | 12.3 ± 4.32 | 12.1 ± 3.82 | 14.5 ± 5.49 | 13.4 ± 4.54 | 13.5 ± 4.66 |
| treated | | | | | | | | | |
| (25 mg/kg) | | | | | | | | | |
| % change | +1.1 | −31.9 | −24.1 | −18.1 | −24.0 | −19.8 | −31.9 | −35.4 | −38.3 |
| compared to | | | | | | | | | |
| control | | | | | | | | | |
| Statistical | ns | ns | ns | ns | ns | ns | ns | ns | ns |
| significance | | | | | | | | | |

TABLE 8
Blood glucose profile of sham and (R)-(−)-7-(3-tert-butylamino-2-hydroxypropoxy)-
3′,5′-dibenzyloxyflavone (4, S-002-853 R) treated db/db mice post oral glucose load.

| | Blood glucose profile (mM) min post glucose load | | | | | |
|---|---|---|---|---|---|---|
| | −30 | 0 | 30 | 60 | 90 | 120 |
| Sham treated | 16.5 ± 8.08 | 21.0 ± 5.81 | 33.1 ± 0.45 | 25.9 ± 0.31 | 21.0 ± 2.68 | 18.4 ± 2.39 |
| S-002-853 (R) treated | 13.6 ± 0.46 | 20.3 ± 4.74 | 34.3 ± 16.9 | 25.9 ± 9.75 | 24.5 ± 9.13 | 23.0 ± 10.0 |
| (25 mg/kg) | | | | | | |
| % change | 17.4 | −3.30 | +3.70 | 0.00 | +17.0 | +24.9 |
| compared to control | | | | | | |

Blood glucose values are Mean ± SE of 3 animals per group. Significance: ns: not significant

TABLE 9
Serum lipid profile in sham and (R)-(−)-7-(3-tert-butylamino-
2-hydroxypropoxy)-3′,5′-dibenzyloxyflavone (4, S-
002-853 R) treated db/db mice.

| | Serum lipid profile (mg/dl) | | |
|---|---|---|---|
| | Triglycerides | Cholesterol | HDL-Chol |
| Sham treated | 147.0 ± 30.6 | 150.0 ± 4.51 | 53.6 ± 4.93 |
| S-002-853 (R) treated | 132.0 ± 11.5 | 144.0 ± 7.37 | 58.0 ± 6.24 |
| (25 mg/kg) | | | |
| % change | −9.72 | −3.72 | +8.06 |
| compared to control | | | |

Values are Mean ± SE of 3 animals per group

(S)-(+)-7-(2-Hydroxy-3-isopropylaminopropoxy)-3′,5′-dihydroxyflavone(11,S-002-857S):

Table 10 presents the blood glucose profile of sham and S-002-857 S treated db/db mice. It is evident from the results that S-002-857 (S) treatment lowered the blood glucose profile of db/db mice. It is evident from the results that 25.0 mg/kg S-002-857 (S) treatment lowered the blood glucose profile from day 4th and continued till the end. An average blood glucose lowering was calculated to be around 20% in this case with S-002-857 (S). The lowering in blood glucose was statistically significant from day 7 to 9 where around 37.4, 36.8 and 43.3% effect was observed. Figure XVII is the graphical presentation of blood glucose profile versus days of treatment at 25 mg/kg dose level. Table 11 and figure XVIII presents the oral glucose tolerance curves of sham treated and S-002-857(S) treated db/db mice. An overall improvement of 27% was evident in the case with S-002-857 (S) at 25.0 mg/kg compared to sham treated group

Table 12 and figure XIX presents the lipid profile of sham and S-002-857 S treated db/db mice. It is evident from the result that 25.0 mg/kg S-002-857 S) treated db/db mice had though lowered serum cholesterol, triglycerides profile compared to sham treated db/db mice but these profiles were not statistically significant. However, an elevation of 17.3% was noted in HDL-cholesterol level in S-002-857 (S) treated db/db mice compared to the sham treated controls.

TABLE 10
Blood glucose profile of sham treated and (S)-(+)-7-(2-Hydroxy-3-isopropyl
aminopropoxy)-3′,5′-dihydroxy flavone (11, S-002-857 S) treated db/db mice.

| | Blood glucose (mM) days post treatment | | | | | | | | |
|---|---|---|---|---|---|---|---|---|---|
| | 1 | 2 | 3 | 4 | 5 | 6 | 7 | 8 | 9 |
| Sham treated | 12.5 ± 1.94 | 12.4 ± 1.90 | 12.5 ± 1.97 | 13.0 ± 2.40 | 13.6 ± 3.43 | 13.0 ± 1.65 | 15.0 ± 1.39 | 14.7 ± 2.69 | 13.3 ± 3.18 |
| S-002-857 (S) | 12.7 ± 2.61 | 12.3 ± 2.40 | 11.5 ± 1.80 | 11.3 ± 2.54 | 10.1 ± 2.08 | 9.19 ± 2.25 | 9.41 ± 1.37 | 9.28 ± 1.02 | 7.56 ± 2.13 |
| treated | | | | | | | | | |
| (25 mg/kg) | | | | | | | | | |
| % change | −1.60 | −0.80 | −8.13 | −12.7 | −25.3 | −29.1 | −37.4 | −36.8 | −43.3 |
| compared to | | | | | | | | | |
| control | | | | | | | | | |
| Statistical | ns | ns | ns | ns | ns | ns | p < 0.05 | p < 0.05 | p < 0.05 |
| significance | | | | | | | | | |

TABLE 11
Blood glucose profile of sham and (S)-(+)-7-(2-Hydroxy-3-isopropyl aminopropoxy)-
3′,5′-dihydroxy flavone (11, S-002-857 S) treated db/db mice post oral glucose load.

| | Blood glucose (mM) min post glucose load | | | | | |
|---|---|---|---|---|---|---|
| | −30 | 0 | 30 | 60 | 90 | 120 |
| Sham treated | 12.9 ± 2.70 | 17.6 ± 5.59 | 33.2 ± 0.19 | 27.2 ± 1.54 | 22.3 ± 0.55 | 18.7 ± 1.64 |
| S-002-857 (S) treated | 8.76 ± 1.90 | 13.8 ± 2.18 | 25.3 ± 0.58 | 19.8 ± 1.20 | 15.2 ± 0.48 | 11.6 ± 0.58 |
| (25 mg/kg) | | | | | | |
| % change | −32.2 | −21.1 | −23.7 | −26.9 | −31.5 | −37.6 |
| compared to control | | | | | | |

TABLE 12
Serum lipid profile in sham and (S)-(+)-7-(2-Hydroxy-
3-isopropyl aminopropoxy)-3′,5′-dihydroxyflavone
(11, S-002-857 S) treated db/db mice.

| | Serum lipid profile (mg/dl) | | |
|---|---|---|---|
| | Triglycerides | Cholesterol | HDL-Chol |
| Sham treated | 133 ± 7.00 | 171 ± 4.73 | 56.0 ± 1.00 |
| S-002-857 (S) | 124 ± 3.51 | 152 ± 7.00 | 65.0 ± 6.03 |
| (25 mg/kg) | | | |
| % change | −6.54 | −11.2 | +17.3 |
| compared to control | | | |

(R)-(−)-7-(2-Hydroxy-3-isopropyl aminopropoxy)-3′,5′-dihydroxy flavone (9, S-002-857 R):

Table 13 presents the blood glucose profile of sham and 25.0 mg/kg S-002-857 R treated db/db mice. A marginal fall in blood glucose profile was observed from day 4th in S-002-857 R treated db/db mice compared to sham treated ones, which more or less remained the same till the end of the study. Figure XX is the graphical presentation of blood glucose profile versus days of treatment of vehicle and S-002-857 R at 25 mg/kg dose level, respectively. There was no significant effect on blood glucose profile was observed on any day post treatment. Table 14 and figure XXI presents the effect of S-002-857 (R) on glucose tolerance of the db/db mice. Though, slight improvement in glucose tolerance was observed in db/db mice treated with 25.0 mg/kg S-002-857 R but it was not found statistically significant.

Table 15 and figure XXII present the lipid profiles of sham and S-002-857 R treated db/db mice. There was found no statistically significant difference between the serum cholesterol, triglycerides and HDL-cholesterol levels of sham and 25.0 mg/kg S-002-857 R treated db/db mice.

TABLE 13
Blood glucose profile of (R)-(−)-7-(2-hydroxy-3-isopropyl
aminopropoxy)-3′,5′-dihydroxyflavone (9, S-002-857
R) treated db/db mice compared to sham treated control.

| | Blood glucose (mM) days post treatment | | | | |
|---|---|---|---|---|---|
| | 1 | 2 | 3 | 4 | 5 |
| Sham treated | 12.5 ± 1.94 | 12.4 ± 1.90 | 12.5 ± 1.97 | 13.0 ± 2.40 | 13.6 ± 3.43 |
| S-002-857 R | 12.5 ± 2.55 | 12.3 ± 2.67 | 12.1 ± 2.02 | 11.5 ± 1.86 | 11.9 ± 2.74 |
| treated | | | | | |
| (25 mg/kg) | | | | | |
| % change | 0.0 | −1.25 | −3.28 | −11.5 | −13.1 |
| compared to | | | | | |
| control | | | | | |
| Statistical | ns | ns | ns | ns | ns |
| significance | | | | | |
| | Blood glucose (mM) days post treatment | | | | |
| | 6 | 7 | 8 | 9 | 10 |
| Sham treated | 13.0 ± 1.65 | 15.0 ± 1.39 | 12.7 ± 2.69 | 13.3 ± 3.18 | 12.9 ± 2.70 |
| S-002-857 R | 11.6 ± 2.73 | 11.7 ± 1.78 | 11.0 ± 0.35 | 11.5 ± 1.00 | 11.9 ± 5.21 |
| treated | | | | | |
| (25 mg/kg) | | | | | |
| % change | −10.7 | −22.0 | −13.4 | −14.0 | −8.30 |
| compared to | | | | | |
| control | | | | | |
| Statistical | ns | ns | ns | ns | ns |
| significance | | | | | |

TABLE 14
Blood glucose profile of sham and (R)-(−)-7-(2-hydroxy-3-isopropyl aminopropoxy)-
3′,5′-dihydroxyflavone (9, S-002-857 R) treated db/db mice post oral glucose load.

| | Blood glucose profile (mM) min post glucose load | | | | | |
|---|---|---|---|---|---|---|
| | −30 | 0 | 30 | 60 | 90 | 120 |
| Sham treated | 12.9 ± 2.70 | 17.6 ± 5.59 | 33.2 ± 0.19 | 27.2 ± 1.54 | 22.3 ± 0.55 | 18.7 ± 1.64 |
| S-002-857 (R) treated | 11.8 ± 5.21 | 16.3 ± 3.91 | 27.6 ± 1.53 | 24.4 ± 2.99 | 20.6 ± 2.08 | 16.0 ± 1.73 |
| (25 mg/kg) | | | | | | |
| % change | −8.31 | −7.05 | −16.7 | −10.0 | −7.39 | −14.3 |
| compared to control | | | | | | |

TABLE 15
Serum lipid profile in sham, and (R)-(−)-7-(2-Hydroxy-
3-isopropyl aminopropoxy)-3′,5′-dihydroxy flavone
(9, S-002-857 R) treated db/db mice.

| | Serum lipid profile (mg/dl) | | |
|---|---|---|---|
| | Triglycerides | Cholesterol | HDL-Chol |
| Sham treated | 133 ± 7.00 | 171 ± 4.73 | 56.0 ± 1.00 |
| S-002-857 (R) treated | 130 ± 3.06 | 160 ± 6.66 | 59.0 ± 2.65 |
| (25 mg/kg) | | | |
| % change | −1.72 | −6.18 | +5.35 |
| compared to control | | | |

Table 16 and 17 presents the comparative antihyperglycemic and antihyperlipidemic profiles of racemic mixture and the two isomers of S-002-853 in db/db mice. It is evident from the data that S-isomer has marked antihyperglycemic and antihyperlipidemic effect compared to racemic mixture at 25.0 mg/kg dose. R-isomer has insignificant antihyperglycemic or antihyperlipidemic effects

TABLE 16
Comparative antihyperlipidemic activity of S-002 853
(S), S-002 853 (R) with S-002-853 (racemic mixture)

| Test | S-002-853 | | | | | | | | |
|---|---|---|---|---|---|---|---|---|---|
| dose | (Racemic) | | | S-002-853 (S) | | | S-002-853 (R) | | |
| (mg/kg) | TG | T-Chol | HDL-C | TG | T-Chol | HDL-C | TG | T-Chol | HDL-C |
| 10 | 13.9* | 8.24 | +3.03^{ns} | 17.9* | 16.3* | +17.4^{ns} | — | — | — |
| 25 | −10.1* | −4.47 | +16.2^{ns} | 14.5** | 27.8* | +33.2* | −9.72^{ns} | −3.72^{ns} | +8.06^{ns} |

Significance
*p < 0.05
**p < 0.01
^{ns}not significant

TABLE 17
Comparative antihyperglycemic activity of S-002 853
(S), S-002 853 (R) with S-002-853 (raceme mixture)

| | S-002-853 | | | | | |
|---|---|---|---|---|---|---|
| Test | (Racemic) | | S-002-853 (S) | | S-002-853 (R) | |
| dose | Average | Day 10 | Average | Day 10 | Average | Day 10 |
| (mg/kg) | Fall | (OGTT) | Fall | (GTT) | Fall | (OGTT) |
| 10 | 44.6* | 15.4* | 42.2* | 27.6* | ND | ND |
| | (day 6) | | (day 8) | | | |
| 25 | 57.7** | 35.8* | 61.1** | 45.3** | 38.3^{ns} | 44.24^{ns} |
| | (day 7) | | (day 6) | | | |

ND: Not done,
Significance
*p < 0.05
**p < 0.01
^{ns}not significant

1. An optically active compound of general formula I and salts thereof: 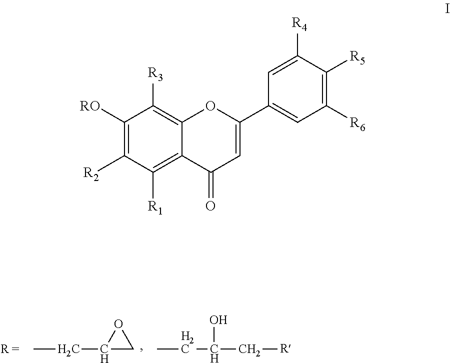
wherein R′ is selected from a group consisting of t-butyl amine, n-butylamine, iso butylamine, iso-propyl amine, 4-phenyl-piperazine-1-ylamine, 4-(2-methoxyphenyl)-piperazin-1-ylamine, and 3,4-dimethoxy phenethyl amine;
wherein R₁, R₂ and R₃ are selected from the group consisting of hydrogen, methyl or iso pentenyl; R₄, R₅ and R₆ are selected from the group consisting of OH, O-alkyl, O-benzyl, O-substituted phenyl or combination thereof,

2. A compound according to claim 1, having formula I wherein the representative compounds comprising:
a) R-(−)-7-(3-tert-Butylamino-2-hydroxy-propoxy)-3′,5′-dibenzyloxy-flavone (4);
b) S-(+)-7-(3-tert-Butylamino-2-hydroxy-propoxy)-3′,5′-dibenzyloxy-flavone (7);
c) R-(−)-7-(3-iso-Propylamino-2-hydroxy-propoxy)-3′,5′-dibenzyloxy-flavone (8);
d) R-(−)-7-(3-iso-Propylamino-2-hydroxy-propoxy)-3′,5′-dihydroxy-flavone (9);
e) S-(+)-7-(3-iso-Propylamino-2-hydroxy-propoxy)-3′,5′-dibenzyloxy-flavone (10);
f) S-(+)-7-(3-iso-Propylamino-2-hydroxy-propoxy)-3′,5′-dihydroxy flavone (11).

3. A compound according to claim 1, wherein the compound of general formula I comprising;

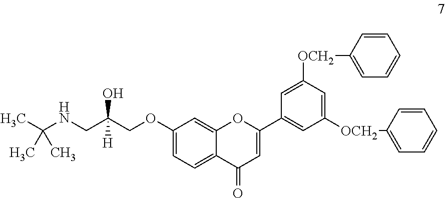

4. A compound according to claim 1, wherein the compound of general formula I comprising;

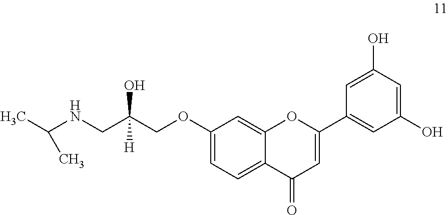

5. A compound according to claim 1, wherein the compound of general formula I comprising;

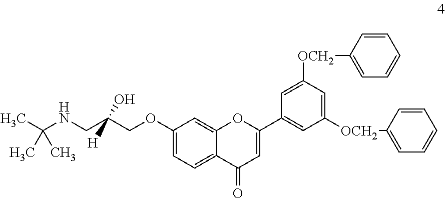

6. A compound according to claim 1, wherein the compound of general formula I comprising;

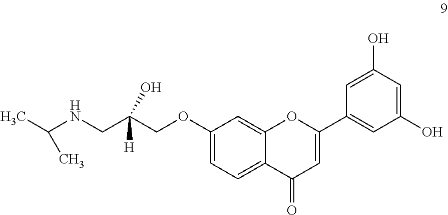

7. A compound according to claim 1, wherein the salt of the general formula I is selected from group consisting of hydrochloride, succinates, fumarates and tartrates.

8. A compound according to claim 1, wherein the compound of general formula I is exhibiting antidiabetic and lipid lowering activity at a dose ranging between 10-250 mg/Kg-body weight.

9. A process for preparation of compound of general formula I according to claim 1, comprising the steps of:
(i) reacting substituted hydroxyflavone of general formula A wherein R₁, R₂ and R₃ are selected from the group consisting of hydrogen, methyl or iso pentenyl; wherein R₄, R₅ and R₆ are selected from the group consisting of O-alkyl, O-benzyl, O-substituted hydroxy, phenyl or combination thereof, with optically active R or S epi-chlorohydrin followed by reaction with a base to obtain the inverted 2,3-epoxy propoxy-flavones of compound of formula B, 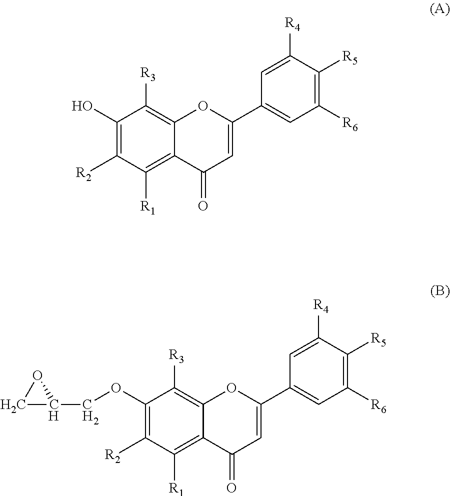
(ii) heating the 2,3-epoxy-propoxy-flavones of the formula B as obtained in step (i) under reflux, with an amine in an alcohol to yield propanolamines of formula C wherein R′ is selected from a group consisting of t-butyl amine, n-butylamine, iso-butylamine, iso-propyl amine, 4-phenyl-piperazine-1-ylamine, 4-(2-methoxyphenyl)-piperazin-1-ylamine, and 3,4-dimethoxy phenethyl amine, 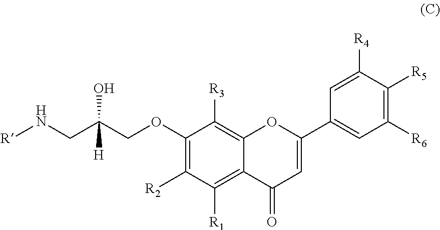
(iii) subjecting the propanolamine obtained in step (ii) for debenzylation using catalytic hydrogenation process to yield corresponding hydroxy flavone derived propanolamines of general formula C wherein R₄, R₅, and R₆ are selected from the group of H, OH and O-methoxy. 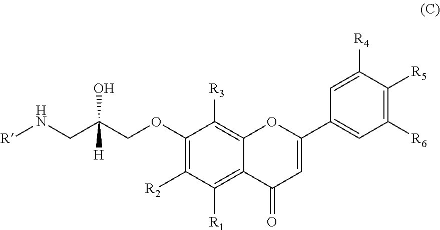
(iv) converting the compound of formula C into salts by known method.

10. A process according to claim 9, wherein the base is selected from the group consisting of sodium hydroxide, potassium hydroxide and benzyltriethylammonium hydroxide.

11. A process according to claim 9, wherein the amine used is selected from the group consisting of alkylamines from a group of cyclic and acyclic alkyl part.

12. A process according to claim 9, wherein the alcohol used is selected from the group consisting of ethanol and propanol.

13. A process according to claim 9, wherein the debenzylation is carried out using Pd—C as catalyst under hydrogen pressure ranging between 40-100 psi.

14. A process according to claim 9, wherein the salt of general formula is selected from the group consisting of hydrochloride, fumarate and tartrates.

15. A method of treating type II diabetes and dislipidemia in mammals, said method comprising the step of administering pharmaceutically effective amount of compound of formula I, and salts thereof according to claim 1, optionally with the pharmaceutically acceptable exciepients.

16. The method according to claim 15, wherein the pharmaceutically effective amount of compound of formula I is in the range of 10-100 mg./kg-body weight.

17. The method according to claim 15, wherein the subject may be selected from animals including human.

18. The method according to claim 15, wherein the compound of formula I showed maximum fall in blood glucose was recorded to be around 61.1% at a dose around 25 mg/kg body weight.

19. The method according to claim 15, wherein the compound (S)-(+)-7-(3-tert-butylamino-2-hydroxypropoxy)-3′,5′-dibenzyloxyflavone (7) showed an elevation of 33.2% was measured in HDL-cholesterol level than the control ones at dose around 25 mg/kg body weight.

20. The method according to claim 15, wherein ED₅₀ of compound of general formula I is in the range of 17.84 to 32.93 mg/kg.

21. The method according to claim 15, wherein the compound (S)-(+)-7-(3-tert-butylamino-2-hydroxypropoxy)-3′,5′-dibenzyloxyflavone (7) enhances HDL-C more than two folds as compared to the racemic compound at a dose of 25 mg/kg body weight.
